(19) United States
(12) Patent Application Publication
(10) Pub. No.: US 2024/0010601 A1
(43) Pub. Date: Jan. 11, 2024

(54) METHODS TO PRODUCE VERY LONG CHAIN FATTY ACIDS (VLCFA)

(71) Applicant: THE UNITED STATES OF AMERICA, AS REPRESENTED BY THE SECRETARY, DEPARTMENT OF HEALTH AND HUMAN, Bethesda, MD (US)

(72) Inventors: Rolf SWENSON, Bethesda, MD (US); Alan REMALEY, Bethesda, MD (US); Zhen-Dan SHI, Bethesda, MD (US); Zhi-hong YANG, Bethesda, MD (US)

(21) Appl. No.: 18/042,743

(22) PCT Filed: Aug. 31, 2021
(86) PCT No.: PCT/US2021/048390

§ 371 Date: Feb. 23, 2023

Related U.S. Application Data
(60) Provisional application No. 63/072,519, filed on Aug. 31, 2020.

(51) Int. Cl. C07C 57/02

(52) U.S. Cl. CPC C07C 57/02

(57) ABSTRACT

The disclosure provides methods of synthesizing very long chain fatty acids, including deuterated very long chain fatty acids. The fatty acids can by polyunsaturated fatty acids. The methods include the step of reacting a protected leaving group (L)-substituted saturated aliphatic group with a halo-substituted unsaturated aliphatic group to form a protected aliphatic group. The protected aliphatic group is deprotected to form an alcohol. The alcohol is then oxidized, thereby forming a very long chain fatty acid.

### CROSS REFERENCE TO RELATED APPLICATIONS

This application claims priority to U.S. Provisional Appl. No. 63/072,519, filed Aug. 31, 2020, which is hereby incorporated by reference in its entirety.

### BACKGROUND

Very long chain fatty acids (VLCFAs) have structurally unusual long hydrocarbon chains (C24-C40). While present in extremely small quantities, VLCFAs are found in a number of species and organs (e.g., testes, retinas, brain, and sperm), and they are essential lipids that play important roles in certain biological systems that cannot be fulfilled by the more common shorter chain C16-C18 fatty acids. Because of their very long chain structure, some VLCFAs are able to span and reside within both leaflets of the lipid bilayer, thereby giving stability to highly curved cellular membranes, such as those which surround nuclear pore complexes. In photoreceptors, the VLC-polyunsaturated FAs (VLCPUFA) are known to be associated with rhodopsin and play a role in regulation of phototransduction cascades. Absence of these VLCPUFAs appears to contribute to macular degeneration in autosomal dominant Stargardt macular dystrophy (STGD3).

Currently VLCPUFA are extracted from natural sources such as bovine retina and fish. Synthetic methods of making VLCPUFAs exist, but these require mercury in the form of aluminum amalgam, which is toxic, and cannot be used in large scale. Extraction provides only minute (microgram) quantities and is a very time consuming and labor intensive process. In addition, although previous works reported biosynthesis of VLCPUFA in cells via overexpressing elongation of very long chain fatty acids 4 (ELOVL4) protein, the technical requirements and very small yield of VLCPUFA products from this method limit its utility.

There exists a need for a method for preparing VLCPUFAs that can provide these therapeutically useful molecules in larger amounts than purification from natural sources and, unlike conventional synthetic methods, avoids the use of toxic mercury. This disclosure fulfills these objectives and provides additional benefits, described herein.

### SUMMARY

The disclosure provides a method for synthesizing very long chain fatty acids.

The method provides step (a) reacting a protected leaving group (L)-substituted saturated aliphatic group (2) with a halo-substituted unsaturated aliphatic group (4) to form a protected aliphatic group (5)

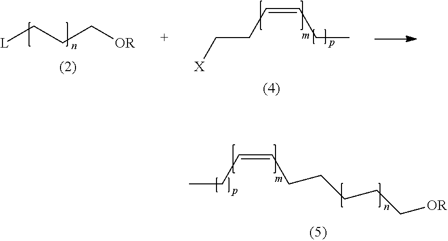

### Wherein

L is a leaving group; X is halogen; n is an integer from 3 to 14; m is an integer from 3 to 6; p is an integer from 1 to 4; and

R is a protecting group.

Next the method includes step (b), deprotecting the protected aliphatic group (5) to form an alcohol (6)

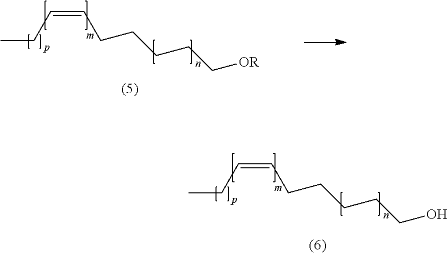

Then in step (c), the method includes oxidizing the alcohol (6) to form a very long chain fatty acid (7);

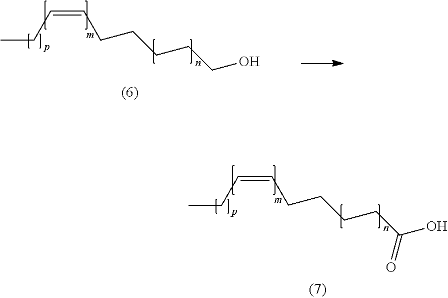

The disclosure also includes very long chain fatty acids with deuteration at the β-position. These fatty acids have the formula

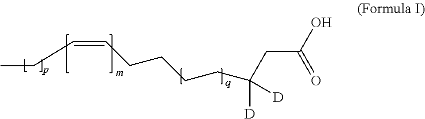

where m is an integer from 3 to 6; p is an integer from 1 to 4; and q is an integer from 2 to 13.

The disclosure includes pharmaceutical compositions comprising a very long chain fatty acid of Formula I together with a pharmaceutically acceptable carrier.

The disclosure includes a method of treating macular degeneration in a patient comprising administering a compound or salt thereof to the patient.

### DETAILED DESCRIPTION

### Chemical Description and Terminology

Compounds are described using standard nomenclature. Unless defined otherwise, all technical and scientific terms used herein have the same meaning as is commonly understood by one of skill in the art to which this invention belongs. Unless clearly contraindicated by the context each compound name includes the free acid or free base form of the compound as well as all pharmaceutically acceptable salts of the compound.

The terms “a” and “an” do not denote a limitation of quantity, but rather denote the presence of at least one of the referenced items. The term “or” means “and/or”. The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the fatty acid synthesis method or the deuterated fatty acids disclosed herein. Unless defined otherwise, technical and scientific terms used herein have the same meaning as is commonly understood by one of skill in the art to which this disclosure belongs.

A dash (“-”) that is not between two letters or symbols is used to indicate a point of attachment for a substituent.

“Halo” or “halogen” means fluorine, chlorine, bromine, or iodine.

“Pharmaceutical compositions” are compositions comprising at least one active agent, such as a compound or salt of Formula I, and at least one other substance, such as a carrier. Pharmaceutical compositions optional contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as hepatitis C.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active compound is provided.

A “pharmaceutically acceptable excipient” means an excipient that is useful in preparing a pharmaceutical composition/combination that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

A “patient” is a human or non-human animal in need of medical treatment. Medical treatment can include treatment of an existing condition, such as a disease or disorder, prophylactic or preventative treatment, or diagnostic treatment. In some embodiments the patient is a human patient.

“Treatment,” as used herein includes providing a compound of Formula (I), either as the only active agent or together with at least one additional active agent sufficient to: (a) prevent a disease or a symptom of a disease from occurring in a patient who may be predisposed to the disease but has not yet been diagnosed as having it (e.g. including macular degeneration in patients having a genetic mutation that predisposes them to macular degeneration such as the autosomal dominant mutation associated with Stargardt muscular dystrophy); (b) inhibiting the disease, i.e. arresting its development; and (c) relieving the disease, i.e., causing regression of the disease. “Treating” and “treatment” also means providing a therapeutically effective amount of a compound of Formula (I), as the only active agent or together with at least one additional active agent to a patient having or susceptible to a condition in which very long chain fatty acids are known to play a role.

“Preventing” a disease or disorder means effecting a statistically significant decrease in the likelihood of developing a disease or disorder in a patient at risk of developing the disease or disorder, or effecting a statistically significant delay in the onset of symptoms or reducing the severity of symptoms in a patient at risk of developing the disease or disorder.

A “therapeutically effective amount” of a pharmaceutical composition/combination of this invention means an amount effective, when administered to a patient, to provide a therapeutic benefit such as an amelioration of symptoms, e.g., an amount effective to decrease the symptoms.

As used herein, the term “administering” means providing a pharmaceutical agent or composition to a subject, and includes, but is not limited to, administering by a medical professional and self-administering.

Fatty acids discussed herein are identified using the following conventional numbering system

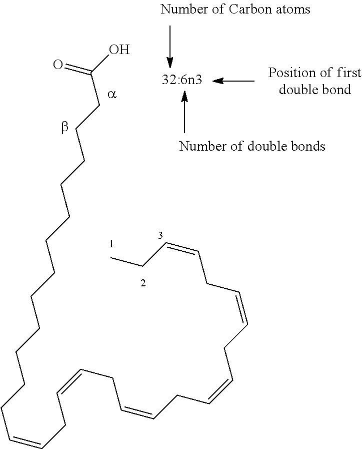 [SMILES: CC/C=C\C/C=C\C/C=C\C/C=C\C/C=C\C/C=C\CCCCCCCCCCCCC(=O)O]

### Embodiments

In an embodiment, provided is a method for synthesizing very long chain fatty acids.

The method provides step (a) reacting a protected leaving group (L)-substituted saturated aliphatic group (2) with a halo-substituted unsaturated aliphatic group (4) to form a protected aliphatic group (5)

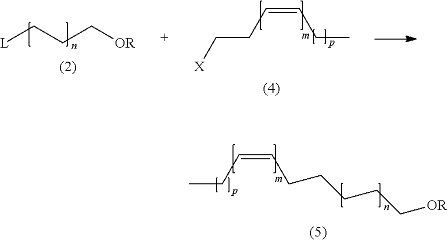

### Wherein

n is an integer from 3 to 14; m is an integer from 3 to 6; p is an integer from 1 to 4; L is a leaving group; X is halogen; and

R is a protecting group.

Next the method includes step (b), deprotecting the protected aliphatic group (5) to form an alcohol (6)

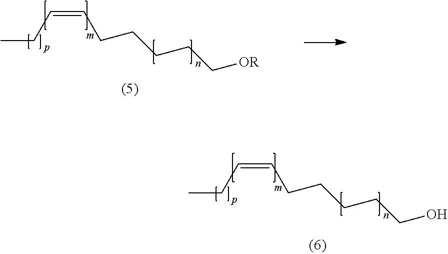

Then in step (c), the method includes oxidizing the alcohol (6) to form a very long chain fatty acid (7);

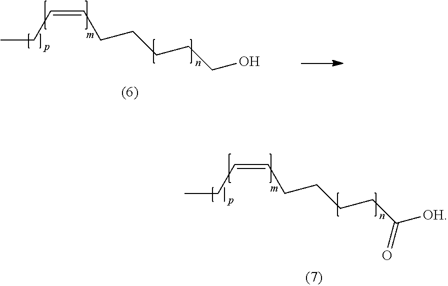

In an embodiment, provided is a method for synthesizing very long chain fatty acids, wherein L may be any suitable leaving group; X may be Cl, Br, or I; and p may be 1 or 4.

In an embodiment, provided is a method for synthesizing very long chain fatty acids,

wherein the leaving group (L) may be an acetoxy (—OC(═O)CH₃), tosyl (—OTs), nosyl (—ONs), arylsulfonyl, Cl, Br, or I, where L and X are not the same.

In an embodiment, X is I and the leaving group (L) is Br.

In an embodiment, provided is a method for synthesizing very long chain fatty acids, wherein X may be Br; and leaving group (L) may be tosyl (—OTs), nosyl (—ONs), Cl, Br, or I.

In an embodiment, provided is a method for synthesizing very long chain fatty acids, wherein X is Br; and leaving group (L) is I.

In an embodiment, the protecting group may be an acetyl, benzoyl, benzyl, β-methoxyethoxyether, methoxymethyl ether, dimethoxytrityl, p-methoxybenzyl ether, p-methoxyphenylether, methythiomethylether, pivaloyl, tetrahydropyranyl, tetrahydrofuranyl, trityl, silyl ethyl, methyl ether, or ethoxyethyl ether protecting group.

In an embodiment, the protecting group may be a silyl ether protecting group selected from a trimethyl silyl group (TMS), a tert-butyldimethylsilyl group (TBDMS), tri-iso-propylsilyloxymethyl group (TOM), triisopropylsilyl group (TIPS), or tert-butyldiphenylsilyl group (TBDPS).

In an embodiment, provided is a method for synthesizing very long chain fatty acids, wherein the protected leaving group (L)-substituted saturated aliphatic group (2) is deuterated at the β-position and the method provides a very long chain fatty acid deuterated at the β-position.

In an embodiment, wherein the very long chain fatty acid (7) is an alpha-linolenic fatty acid chosen from a 22:6n3, 24:6n3, 26:6n3, 28:6n3, 30:6n3, 32:6n3, 34:6n3, 36:6n3, 38:6n3, 18:3n3, 18:4n3, 20:4n3, 20:5n3, 22:5n3, 24:5n3, 26:5n3, 28:5n3, 30:5n3, 32:5n3, 34:5n3, 36:5n3, or 38:5n3 fatty acid.

In an embodiment, wherein the very long chain fatty acid (7) is an alpha-linolenic fatty acid chosen from a 32:6n3, 34:6n3, 32:5n3, or 34:5n3 fatty acid.

In an embodiment, wherein the very long chain fatty acid (7) is a linolenic fatty acid chosen from a 18:2n6, 18:3n6, 20:3n6, 20:4n6, 22:4n6, 24:4n6, 26:4n6, 28:4n6, 30:4n6, 32:4n6, 34:4n6, 36:4n6, 38:4n6, 22:5n6, 24:5n6, 26:5n6, 28:5n6, 30:5n6, 32:5n6, 34:5n6, 36:5n6, or 38:5n6 fatty acid.

In an embodiment, wherein the very long chain fatty acid (7) is a linolenic fatty acid chosen from a 28:4n6, 34:4n6, 36:4n6, 30:5n6, or 34:5n6 fatty acid.

In an embodiment, provided is a very long chain fatty acid with deuteration at the β-position of Formula I,

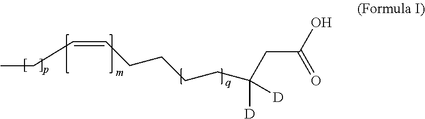

wherein m is an integer from 3 to 6; p is an integer from 1 to 4; and q is an integer from 2 to 13. The level of deuteration at each position indicated with a “D” in Formula I can be greater than 50%, greater than 60%, greater than 70%, greater than 80%, greater than 90%, greater than 95%, greater than 98%, or greater than 99%.

In an embodiment, Formula I includes all pharmaceutically acceptable salts of Formula I.

### Pharmaceutical Compositions

Provided herein are pharmaceutical compositions comprising a very long chain fatty acid of Formula I together with a pharmaceutically acceptable carrier.

The pharmaceutical composition may have any suitable form, and may be a tablet, capsule, lyophilized solid, solution, suspension, or a combination thereof. The pharmaceutical composition may be an intravenous, injectable, topical, or oral dosage form. The pharmaceutical composition may be a dosage form intended for parenteral administration, such a lyophilized solid needing reconstitution before administration or a reconstituted solution of the lyophilized solid. The pharmaceutical composition can be an ocular formulation, such as a liquid topical formulation. The pharmaceutical composition may be an oral dosage form in the form of a tablet or capsule. In a preferred embodiment, the VLCFA is formulated into any oral dosage form including solid, semi-solid, liquid, powder, sachet and the like. Solid oral dosage forms can include, for example, a tablet, a capsule (hard or soft), or subunits, and the like. “Subunit” includes a minitablet, a bead, a spheroid, a microsphere, a seed, a pellet, a caplet, a microcapsule, a granule, a particle, and the like that can provide an oral dosage form alone or when combined with other subunits. Exemplary semi-solid or liquid dosage forms include a suspension, a solution, an emulsion, and the like. Solid oral dosage forms can also include orally dissolving/disintegrating dosage (ODT) forms. Exemplary ODTs include orally dissolving/disintegrating tablets, orally dissolving films and dosage forms intended for sublingual/lingual/buccal delivery such as fast dissolving/disintegrating sublingual tablets and films.

The oral dosage form can be formulated for a specific type of release including immediate-release, controlled-release, sustained-release, or extended-release.

The disclosure includes pharmaceutical compositions comprising a compound of Formula I or a salt thereof.

The disclosure includes methods in which one or more compounds are an admixture or otherwise combined with one or more compounds and may be in the presence or absence of commonly used excipients (or “pharmaceutically acceptable carriers”); for example, but not limited to: i) diluents and carriers such as starch, mannitol, lactose, dextrose, sucrose, sorbitol, cellulose, or the like; ii) binders such as starch paste, gelatin, magnesium aluminum silicate, methylcellulose, alginates, gelatin, sodium carboxymethyl-cellulose, polyvinylpyrrolidone or the like; iii) lubricants such as stearic acid, talcum, silica, polyethylene glycol, polypropylene glycol or the like; iv) absorbents, colorants, sweeteners or the like; v) disintegrates, (e.g., calcium carbonate and sodium bicarbonate) such as effervescent mixtures or the like; vi) excipients (e.g. cyclodextrins or the like); vii) surface active agents (e.g., cetyl alcohol, glycerol monostearate), adsorptive carriers (e.g., kaolin and bentonite), emulsifiers or the like. Examples of carriers include, without limitation, any liquids, liquid crystals, solids or semi-solids, such as water or saline, gels, creams, salves, solvents, diluents, fluid ointment bases, ointments, pastes, implants, liposomes, micelles, giant micelles, or the like, which are suitable for use in the compositions.

Furthermore, the disclosure includes compositions prepared using conventional mixing, granulating, or coating methods and may contain 0.01 to 90% of the active ingredients. The resulting compositions (formulations) may be presented in unit dosage form and may be prepared by methods known in the art of pharmacy. All methodology includes the act of bringing the active ingredient(s) into association with the carrier which constitutes one or more ingredients. Therefore, compositions (formulations) are prepared by blending active ingredient(s) with a liquid carrier or a finely divided solid carrier, and/or both, and then, if needed, shaping the product into a desired formulation.

Certain compositions of the disclosure contain compound from about 90 to about 80% by weight, from about 80 to about 70% by weight, from about 70 to about 60% by weight, from about 60 to about 50% by weight, from about 50 to about 40% by weight, from about 40 to about 30% by weight, from about 30 to 20% by weight, from about 20 to about 10% by weight, from about 10 to about 4% by weight, from about 4.0% to about 2.0% by weight, from about 2.0% to about 1.0% by weight, and even from about 1.0% to about 0.01% by weight. The effective amount of oral and intravenous compounds or compositions of the disclosure may range from about 0.1 to 100 milligrams (mg) per kilogram (kg) of subject weight. In certain embodiments, the compounds or compositions of the disclosure are administered at from about 0.0001 mg/kg to 0.1 mg/kg (e.g. diagnostic monitoring), or from 0.1 mg/kg to 2 mg/kg, or from about 2 mg/kg to 5 mg/kg; in other embodiments, from about mg/kg to 10 mg/kg, from about 10 mg/kg to 20 mg/kg, from about 20 mg/kg to 30 mg/kg, from about 30 mg/kg to 40 mg/kg, from about 40 mg/kg to 50 mg/kg, from about 50 mg/kg to mg/kg or from about 75 mg/kg to 100 mg/kg.

The disclosure includes ophthalmic compositions. The disclosed compositions can be emulsions, solutions, suspensions, gels, ointments, occlusive films, or a sustained release films and they can be preserved or non-preserved formulations. The compositions can be formulated as eye drops, creams, ointments, and films that can be applied to an eye. The formulations can be administered to the eye, the upper eye lid, the lower eye lid, or a combination thereof. Topical administration of the compositions provides treatment at the site of the condition with minimal systemic levels of the VLEFA of the disclosure.

Ophthalmic compositions can include polymeric emulsifiers, such as castor oil, squalene, isosterate, and isopropyl myristate; lipophilic components, such as mineral oil, silicone oil, caprylic/capric triglycerides, Alcohols, such as cetyl alcohols and stearyl alcohols. Ophthalmic compositions of the disclosure may also contain diethylene glycol monoethyl ether, propylene glycol, dipropylene glycol; Co-solvents such as dimethyl ether, diethylene glycol, and dipropylene glycol; buffers and pH modifying agents, such as sodium citrate dihydrate, boric acid, monosodium phosphate, dibasic heptahydrate, and sodium phosphate monobasic monohydrate. Ophthalmic compositions can contain cyclodextrin, hydroxypropyl-beta-cyclodextrin, hydroxyethyl cellulose, PEG 300, PEG 400, povidone, glycerin, propylene glycol, and hydroxypropyl methyl cellulose. Suitable preservatives include benzalkonium chloride. The ophthalmic composition can contain a plasticizer or a film former. Ophthalmic compositions typically contain a vehicle such as water, 0-99% wt/vol.

The frequency, duration, and dosage of the administration are determined by the prescribing physician. The dosage can vary depending on the dosage form. When the composition is a solution, for example, 1, 2, 3, or more drops can be administered per eye per administration. Frequency of administration can be one or more times daily (such as once, twice, three, or four or more times daily), bi-weekly, and/or monthly. Duration of administration can continue until the condition to be treated is resolved, that is, until one or more symptoms of the ocular condition are reduced or eliminated. Accordingly, the composition can be administered for hours, days, weeks, months, and years.

A symptom is alleviated if it is prevented, reduced or eliminated. A symptom is prevented in a patient that typically experiences a particular symptom with the ocular condition (or if patients similarly situated typically experience a particular symptom) and the patient does not experience the onset of the symptom following administration of the disclosed composition. A reduction of a symptom is considered achieved if there is a 5%, 10%, 20%, 50%, 75%, 90% or more reduction in the severity or duration of one or more symptom associated with the ocular condition, in a patient. An elimination of one or more symptoms associated with the ocular condition is achieved when it ceases to be present or substantially present in a patient.

The ingredients particularly mentioned above are merely examples and that some embodiments of formulations comprising the compositions of the present disclosure include other suitable components and agents. The invention further includes packages, vessels, or any other type of container that contain a compound of the present invention.

### Methods of Treatment

The disclosure includes a method of treating a disease or disorder responsive to treatment with a very long chain fatty acid in a patient comprising administering a very long chain fatty acid to the patient. In an embodiment the disclosure includes a method of treating macular degeneration in a patient comprising administering a very long chain fatty acid of the disclosure, including a deuterated very long chain fatty acid, or salt thereof, to the patient. The disclosure includes a method of treating or preventing other diseases and disorders associated with very long chain fatty acids such as 3-hydroxyacyl-CoA dehydrogenase deficiency, mitochondrial trifunctional protein deficiency, very long-chain acyl-CoA dehydrogenase deficiency. The disclosure further includes methods of treating inflammatory disorders comprising administering an effective amount of a VLCFA of the disclosure, such as a deuterated VLCFA of the disclosure, to a patient having an inflammatory disorder. A method of treating or preventing macular degeneration in a patient comprising administering an effective amount of a compound of any one of claims 1 to 14 to the patient. The disclosure includes a method of reducing, treating, or preventing any of the following in a patient: heart failure, type II diabetes, cardiovascular disease, cardiac arrhythmia, cognitive decline, cancer, or hypertension by administering an effective amount of a VLCFA, such as a VLCFA prepared by a method of the disclosure. The cardiac arrhythmia may be atrial fibrillation or premature ventricular contractions. The VLCFA can be a deuterated VLCFA, such as a VLCFA with B-position deuteration. The VLCFA can be a polyunsaturated VLCFA, including a polyunsaturated deuterated VLCFA.

The VLCFA of the disclosure can be administered by any acceptable means of pharmaceutical administration. Examples of methods of administration include, but are not limited to, oral administration (e.g., ingestion, buccal or sublingual administration), anal or rectal administration, topical application, aerosol application, inhalation, intraperitoneal administration, intravenous administration, transdermal administration, intradermal administration, subdermal administration, intramuscular administration, intrauterine administration, vaginal administration, administration into a body cavity, surgical administration, administration into the lumen or parenchyma of an organ, and parenteral administration. The compositions can be administered in any form by any means. Examples of forms of administration include, but are not limited to, injections, solutions, creams, gels, implants, ointments, emulsions, suspensions, microspheres, powders, particles, microparticles, nanoparticles, liposomes, pastes, patches, capsules, suppositories, tablets, transdermal delivery devices, sprays, suppositories, aerosols, or other means familiar to one of ordinary skill in the art.

In some embodiments, the VLCFA, or compositions of the present disclosure, are administered to persons or animals to provide substances in any dose range that will produce desired physiological or pharmacological results. Dosage will depend upon the substance or substances administered, the therapeutic endpoint desired, the diagnostic endpoint desired, the desired effective concentration at the site of action or in a body fluid, and the type of administration. In some embodiments, the compounds and compositions of the present disclosure may be administered to a subject. In certain embodiments, the subject is a mammal. Embodiments in which the subject is a human patient are within the scope of the disclosure. Embodiments in which the subject is a companion animal, e.g. cat, dog, or a livestock animal, e.g. sheep, bovine, or swine, are within the scope of the disclosure.

A therapeutically effective amount includes an amount that, upon administration to a patient, results in a discernible patient benefit. An effective amount of a VLCFA composition can range from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/g and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents. The VLCFA may administered once, but will more typically be administered 1 or more times per day for a period of days, weeks, months, or indefinitely. It can be administered a daily supplement. In an embodiment the VLCFA is administered 1 or more times per day, indefinitely, to a human subject or human patient above the age of 40, 45, 50, 55, 60, 65, 70, 75, or older to promote healthy aging and lower the risk of unhealthy aging events. A total of 20 mg, 50 mg, 100 mg, 200 mg, 300 mg, 400 mg, 500 mg, 600 mg, 700 mg, 800 mg, 900 mg, or 1000 mg can be administered to the patient or subject daily.

### Combination Methods of Treatment

The disclosure includes methods in which the fatty acid of the disclosure is the only active agent or in which the fatty acid of the disclosure, such as a deuterated fatty acid of the disclosure, is administered in combination with at least one additional active agent. For treatment of macular degeneration, the additional active agent may be an alpha agonist, a beta blocker, a carbonic anhydrase inhibitor (CAI), a cholinergic drug, a prostaglandin analog, a Rho kinase inhibitor, or a combination of the any of the foregoing. Suitable alpha agonists include Brimonidine tartrate (ALPHAGAN P) and apraclonidine (LOPIDINE). Beta blockers include timolol, levobunolol (BETAGAN), metipranolol (OPTIPRANOLOL), Betaxolol (BETOPTIC S). Carbonic anhydrase inhibitors (CAIs) include dorzolamide (TRUSOPT), brinzolamide (AZOPT), acetazolamide (DIAMOX), methazolamide (NEPTAZANE). Cholinergic or Miotic medications include pilocarpine and carbachol. Prostaglandin analogs include latanoprost (XALATAN), bimatoprost (LUMIGAN), travoprost (TRAVATAN Z), tafluprost (ZIOPTAN), and latanoprostene bunod (VYZULTA). Rho kinase inhibitors include netarsudil (RHOPRESSA). Combined medications include COSOPT, a combination of a beta blocker (timolol) and a carbonic anhydrase inhibitor (dorzolamide); COMBIGAN, a combination of an alpha agonist (brimonidine) and a betal blocker (timolol); SIMBRINZA, a combination of brinzolamide and brimonidine; ROCKLATAN, a combination of a Rho kinase inhibitor netarsudil and the prostaglandin analog, latanoprost.

Hereinafter, one or more embodiments will now be described in more detail with reference to the following examples. However, these examples are provided herein for illustrative purpose only and not intended to limit the scope of the one or more embodiments.

### EXAMPLES

### Abbreviations

DCM dichloromethane
DHP 3,4-dihydropyran
DMF dimethyl formamide
PPTS pyridinium p-toluensulfonate
p-TSOH p-toluene sulfonic acid
TBDPS tert-butyldiphenylsilyl protecting group
THF tetrahydrofuran
THP tetrahydropyranyl ether
TBAF tetra-n-butylammonium fluoride
TBDPS tert-butyldiphenylsilyl

### Example 1. Synthesis of Very Long Chain Fatty Acid, Alpha-Linolenic Acid 32:6N3

The very long chain fatty acids of the disclosure can be prepared according to Scheme 1. The preparation of Compound 7-a is exemplified. One of ordinary skill will recognize changes in starting materials that may be made to prepare other long chain fatty acids.

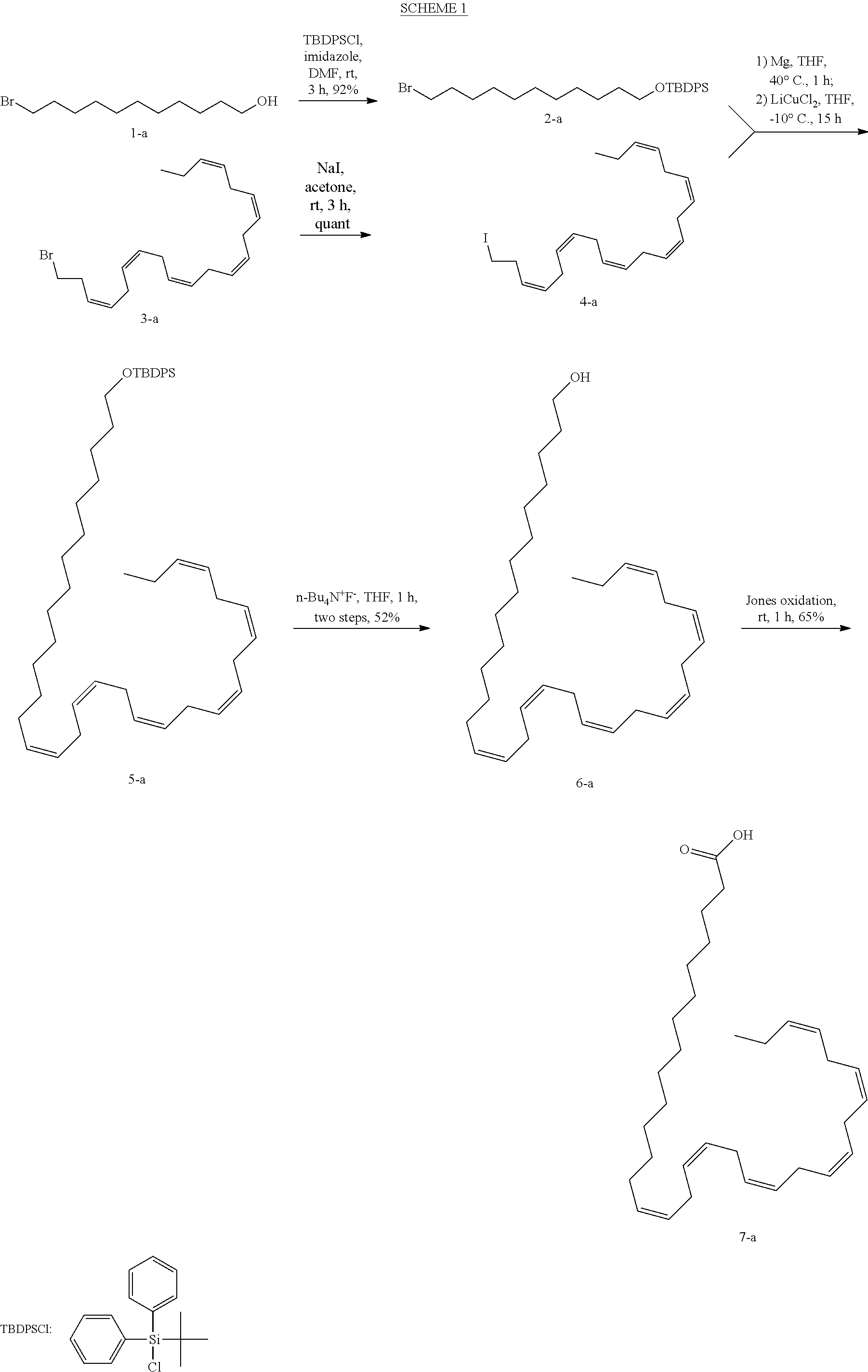

### Overview

Synthesis of very-long-chain fatty acids (VLCFA) compound 7-a is outlined in scheme 1. 10-bromodecan-1-ol 1 was protected to form tert-butyldiphenylsilyl (TBDPS) ether 2-a in 92% yield. Exchange of the bromo group of (3Z,6Z,9Z,12Z,15Z,18Z)-1-bromohenicosa-3,6,9,12,15,18-hexaene 3-a with sodium iodide produced compound 4-a in quantitative yield. Compound 2-a reacted with magnesium to form the Grignard reagent, which was used for coupling with compound 4-a in the presence of LiCuCl₂ to afford the key intermediate 5-a. After deprotection of the TBDPS group with n-Bu4N⁺F⁻, followed by Jones oxidation, the final VLCFA 7-a was obtained in 33% yield in three steps. This method is used for preparation of a series of VLCFA derivatives. The method provided in Scheme I is facile and avoids the use of toxic chemicals such as mercury (Hg) common in methods known in the art. The method of Scheme I should be easily scalable.

### Step 1: Preparation of Bromo-TBDPS Alkane (Compound 2-a)

A halogenated alcohol (1-a) (1.72 grams (g), 6.86 millimoles (mmol)), tert-butyldiphenylsilyl chloride (TBDPSCl) (10.29 mmol, 1.5 equivalents (eq.)), and imidazole (1.401 g, 20.59 mmol, 3.0 eq.) are dissolved in dry DMF (10 milliliters (mL)) under nitrogen and the resulting solution is stirred for 3 hours (hr) at room temperature (RT) at which point, water (15 mL) and diethyl ether (25 mL) were added, the two layers were separated, and the water layer is extracted with diethyl ether (3×25 mL). The combined organic extracts are washed with water, brine, dried over MgSO₄, filtered and evaporated. The pure product from the crude is isolated by Flash silica gel column chromatography (elution with 100% hexane to 5% ethyl acetate/hexane) to furnish Compound 2-a as a clear thick oil.

### Step 2: Preparation of Compound 5-a

### (a) Preparation of Iodoalkene (Compound 4-a)

Bromoalkene 3-a (5 mmol) is treated with NaI (50 mmol) in acetone (10 mL) at RT for 3 hr. The reaction mixture is concentrated in vacuum. Water (15 mL) and diethyl ether (25 mL) are added, the two layers are separated, and the water layer is extracted with diethyl ether (3×25 mL). The combined organic extracts are washed with water, brine, dried over MgSO₄, filtered and evaporated to afford iodoalkene (Compound 4-a) in a quantitative yield.

### (b) Preparation of Compound 5-a

To magnesium (5 mmol) in 10 mL of THF is added over 10 minutes, 5 mmol of Compound 2-a dissolved in 10 mL of THF. The resulting mixture is stirred for 30 minutes and then cooled to −10° C. To this cooled mixture LiCuCl₂ (6 mmol) is added. Stirring of the mixture at a temperature under −10° C. is continued. A solution of Compound 4-a (4 mmol) in 5 mL of THF is then added dropwise. After the addition is complete, the mixture is stirred for 15 hours and then quenched by being poured into 10 mL of cold saturated aqueous ammonium chloride solution. The solution is extracted with ether and the organic layers are combined, dried, and concentrated to afford a crude Compound 5-a, which is used as is for the next step.

### Step 3: Preparation of Compound 6-a

To a solution of Compound 5-a (5 mmol) in 10 mL of THF is added 5 mmol of tetrabutylammonium fluoride (TBAF) solution (1 molar (M) in THF). The resulting mixture is stirred for 1 hr at RT at which point, water (15 mL) and diethyl ether (25 mL) are added, the two layers are separated, and the water layer is extracted with diethyl ether (3×25 mL). The combined organic extracts are washed with water, brine, dried over MgSO₄, filtered and evaporated. The pure product from the crude is isolated by Flash silica gel column chromatography (elution with 100% hexane to 15% ethyl acetate/hexane) to furnish alcohol, Compound 6-a.

### Step 4: Preparation of Compound 7-a

The preparation of the Jones reagent.

In a 500 mL beaker, chromium trioxide (25 g, 0.25 mole (mol)) is dissolved in distilled water (75 mL), the resulting solution is cooled in an ice-water bath, concentrated sulfuric acid (25 mL) is added slowly with careful stirring. The temperature of the solution is maintained between 0 and 5° C. The concentration of the solution prepared by this procedure is ˜2.5 M.

Oxidation of Compound 6-a

To a solution of Compound 6-a (5 mmol) in 10 mL of acetone is added 6.7 mmlol of Jone's reagent (2.5 M in water) at RT. The rate of addition is adjusted such that the temperature of the reaction mixture did not rise above 35° C. The resulting mixture is stirred for 1 hr at RT at which point, water (15 mL) and ethyl acetate (25 mL) were added, the two layers are separated, and the water layer is extracted with ethyl acetate (3×25 mL). The combined organic extracts are washed with water, brine, dried over MgSO₄, filtered and evaporated. The pure product from the crude is isolated by Flash silica gel column chromatography (elution with 100% hexane to 40% ethyl acetate/hexane) to furnish acid, Compound 7-a (65%).

### Example 2. Synthesis of Very Long Chain Fatty Acids with B-Position Deuteration

Very long chain fatty acids with deuteration at the beta (β)-position can be prepared according to the following scheme. The preparation of 32:6n3 alpha-linolenic is exemplified. One of ordinary skill will recognize changes in starting materials that may be made to prepare other fatty acids with beta-position deuteration.

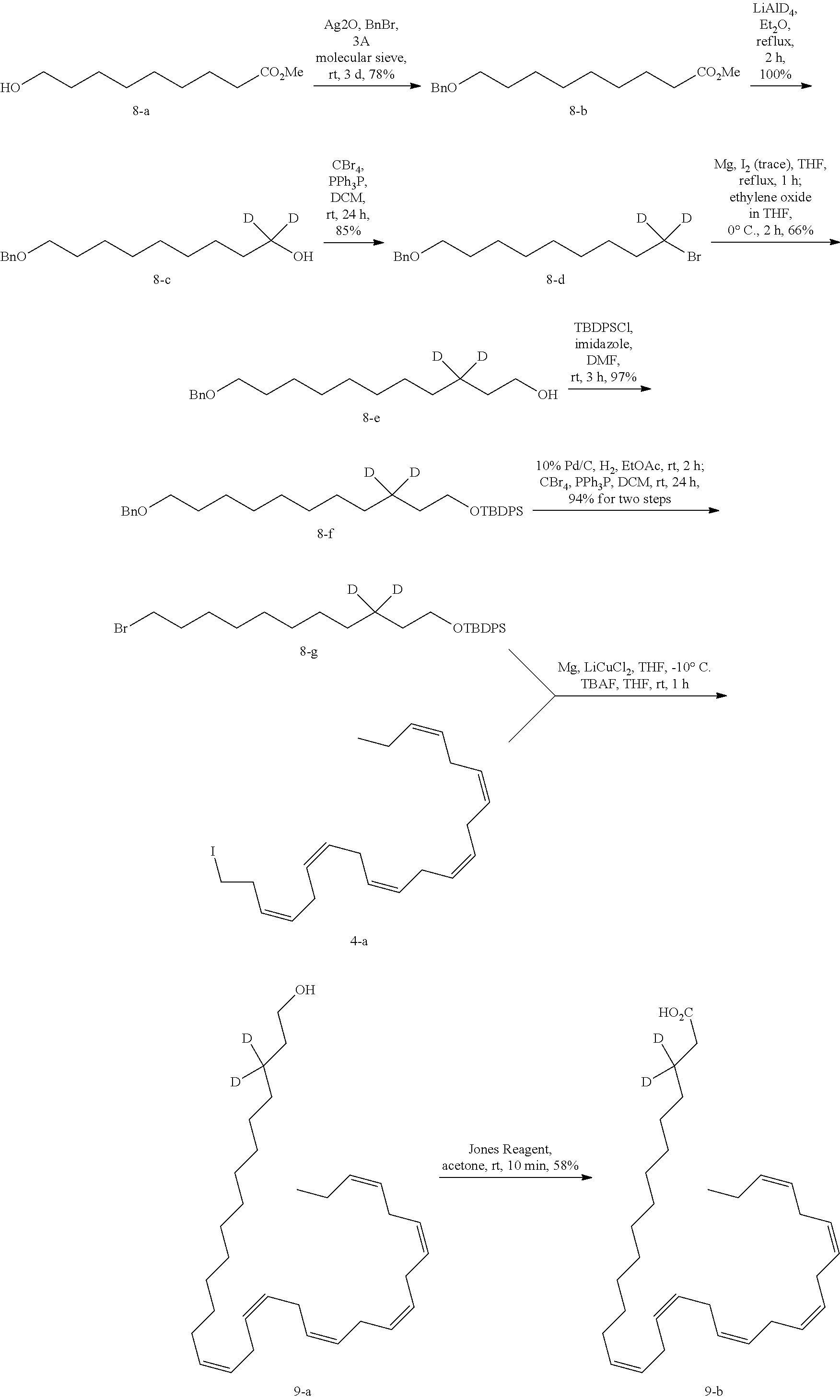

### Step 1: Preparation of methyl 9-(benzyloxy)nonanoate (Compound 8-b)

To compound 8-a (3.77 g, 20.1 mmol) were successively added benzyl bromide (4.76 mL, 40.2 mmol), silver oxide (6.98 g, 30.1 mmol) and 3 Å molecular sieves (6.0 g). The reaction mixture was stirred at room temperature for 3 days before being filtrated through a celite pad. After abundant washing of the celite pad with CH₂C12, the filtrate was concentrated under rotary evaporation. The crude was purified by silica gel flash chromatography to afford compound 8-b (4.35 g, 78% yield). ¹H NMR (CDCl₃): δ 7.34-7.26 (m, 4.45 (s, 2H), 3.67 (s, 3H), 3.46 (t, 2H, J=6.7 Hz), 2.30 (t, 2H, J=7.6 Hz), 1.64-1.53 (m, 1.40-1.23 (m, 7H). MS (ESI) 279 (M+1).

### Step 2: Preparation of 9-(benzyloxy)nonan-1,1-d2-1-ol (Compound 8-c)

Ester (Compound 8-b) (4.32 g, 15.5 mmol) was dissolved in dry diethyl ether (70 mL) under argon atmosphere. LiAlD₄ (1.5 g, 36.6 mmol) was added slowly to the above solution. The resulting reaction mixture was refluxed for 2 h and cooled in ice-water bath. Water (20 mL), 10% NaOH (20 mL) and diethyl ether (50 mL) were added. The water layer was extracted with diethyl ether (3×50 mL). The combined organic extracts were washed with brine and dried over MgSO₄. After evaporation of solvents, the residue was purified by silica gel flash chromatography to afford compound 8-c (3.91 g, 100% yield). ¹H NMR (CDCl₃): δ 7.36-7.33 (m, 5H), 4.50 (s, 2H), 3.46 (t, 2H, J=6.7 Hz), 2.30 (t, 2H, J=7.6 Hz), 1.63-1.51 (m, 4H), 1.40-1.24 (m, 10H). MS (ESI) 253 (M+1).

### Step 3: Preparation of (((9-bromononyl-9,9-d2)oxy)methyl)benzene (Compound 8-d)

A solution of compound 8-c (3.91 g, 15.5 mmol) and carbon tetrabromide (5.77 g, 17.2 mmol) in dichloromethane (20 mL) was cooled to 0° C. A solution of triphenyl phosphine (4.56 g, 17.2 mmol) in dichloromethane (30 mL) was added dropwise to the above solution. The resulting solution was stirred at RT for 24 h. The mixture was concentrated (around 10 mL) and quickly added to stirring hexane (0.5 L). The white precipitate was filtered, and the remaining solution was concentrated and purified by silica gel flash chromatography to afford compound 8-d (4.15 g, 85% yield). ¹H NMR (CDCl₃): δ 7.35-7.26 (m, 5H), 4.50 (s, 2H), 3.46 (t, 2H, J=6.5 Hz), 1.84 ((t, 2H, J=7.4 Hz), 1.65-1.58 (m, 2H), 1.46-1.26 (m, 10H).

### Step 4: Preparation of 11-(benzyloxy)undecan-3,3-d2-1-ol (Compound 8-e)

A dried flask was charged with magnesium turnings (726 mg, 30.3 mmol) and a trace amount of iodine under argon atmosphere. Anhydrous THF (15 mL) was added. Then a solution of compound 8-d (3.83 g, 12.1 mmol) in anhydrous THF (15 mL) was added dropwise to the reaction flask while the reaction mixture remained boiling. The reaction mixture was refluxed for additional 1 h to afford a Grignard solution, which was cooled to 0° C. The Grignard solution was transferred to a dried flask charged with CuI (230 mg, 1.21 mmol) under argon atmosphere. To the above mixture was added ethylene oxide THF solution (16.5 mL, 41.1 mmol) and the stirring was continued for an additional 2 h at 0° C. Then water (30 ml) was added and the aqueous layer was extracted with diethyl ether (3×100 mL). The organic phases were combined, washed with brine and dried over MgSO₄. After evaporation of solvents, the residue was purified by silica gel flash chromatography to afford compound 8-e (2.23 g, 66% yield). ¹H NMR (CDCl₃): δ 7.35-7.26 (m, 5H), 4.50 (s, 2H), 3.63 (t, 2H, J=6.7 Hz), 3.46 (t, 2H, J=6.7 Hz), 1.63-1.54 (m, 4H), 1.40-1.20 (m, 12H). MS (ESI) 303 (M+Na⁺).

### Step 5: Preparation of 11-(benzyloxy)undecan-3,3-d2-1-ol (Compound 8-f)

Compound 8-f was prepared by following the same procedure as in Step 1 of Example 1 to prepare Compound 2-a, except Compound 8-e (2.49 g, 8.89 mmol) was used as an alcohol instead of Compound 1-a, to afford Compound 8-f (4.47 g, 97% yield). ¹H NMR (CDCl₃): δ 7.69-7.65 (m, 4H), 7.42-7.33 (m, 11H), 4.50 (s, 2H), 3.65 (t, 2H, J=6.6 Hz), 3.46 (t, 2H, J=6.6 Hz), 1.66-1.50 (m, 4H), 1.40-1.22 (m, 12H). MS (ESI) 521 (M+Na⁺).

### Step 6: Preparation of ((11-bromoundecyl-3,3-d2)oxy)(tert-butyl)diphenylsilane (Compound 8-g)

(a) Removal of Benzyl Protecting Group

10% Pd/C (2.0 g) was added to a solution of compound 8-f (4.60 g, 8.89 mmol) in ethyl acetate (60 mL) and ethanol (60 mL). The mixture was stirred under hydrogen at atmospheric pressure at RT for 2 h. The mixture was filtered on celite, and the filtrate was concentrated under reduced pressure to afford the corresponding alcohol (3.80 g, 100% yield) which was used in next step without any further purification.

(b) Preparation of Compound 8-g

Compound 8-g is prepared by following the same procedure as in Step 3 of Example 2 to prepare Compound 8-d, except alcohol obtained in Step 6(a) (3.80 g, 8.89 mmol) was used instead of Compound 8-c to furnish Compound 8-e (4.01 g, 94% yield). ¹H NMR (CDCl₃): δ 7.69-7.65 (m, 4H), 7.44-7.35 (m, 6H), 3.65 (t, 2H, J=6.7 Hz), 3.41 (t, 2H, J=6.8 Hz), 1.89-1.82 (m, 2H), 1.55-1.52 (m, 2H), 1.46-1.38 (m, 2H) 1.34-1.22 (m, 10H).

### Step 7: Preparation of (14Z,17Z,20Z,23Z,26Z,29Z)-dotriaconta-14,17,20,23,26,29-hexaen-3,3-d2-1-ol (Compound 9-a)

(a) Copper (I) Mediated Coupling Step

Protected compound 9-a is prepared by following the same procedure as in Step 2 of Example 1 to prepare compound 5-a, except compound 8-g (80 mg) was used instead of compound 2-a to afford a mixture of coupling product which was used in next step without any further purification.

(b) Deprotection of TBDPS Protecting Group

Compound 9-a prepared by following the same procedure as in Step 3 of Example 1 to prepare compound 6-a, except a mixture of coupling product was used instead of compound 5-a to afford corresponding alcohol (5.0 mg). ¹H NMR (CDCl₃): δ 5.40-5.33 (m, 12H), 3.64 (t, 2H, J=6.7 Hz), 2.86-2.80 (m, 10H), 2.12-2.02 (m, 4H), 1.57-1.53 (m, 4H), 1.38-1.20 (m, 16H), 0.97 (t, 2H, J=7.6 Hz). MS (ESI) 479 (M+Na⁺).

### Step 8: Preparation of (14Z,17Z,20Z,23Z,26Z,29Z)-dotriaconta-14,17,20,23,26,29-hexaenoic-3,3-d2 acid (Compound 9-b)

Compound 9-b is prepared by following the same procedure as in Step 4 of Example 1 to prepare Compound 7-a, except alcohol (5.0 mg) obtained in Step 8 was used instead of Compound 6-a to afford Compound 9-b (3.0 mg, 58% yield). ¹H NMR (CDCl₃): δ 5.41-5.33 (m, 12H), 2.85-2.80 (m, 10H), 2.35 (m, 1H), 2.33 (s, 2H), 2.10-2.02 (m, 3H), 1.68-1.20 (m, 18H), 0.97 (t, 2H, J=7.4 Hz). MS (ESI) 493 (M+Na⁺).

### Example 3. Synthesis of VLCFA with B-Position Deuteration Using a THP Protecting Group

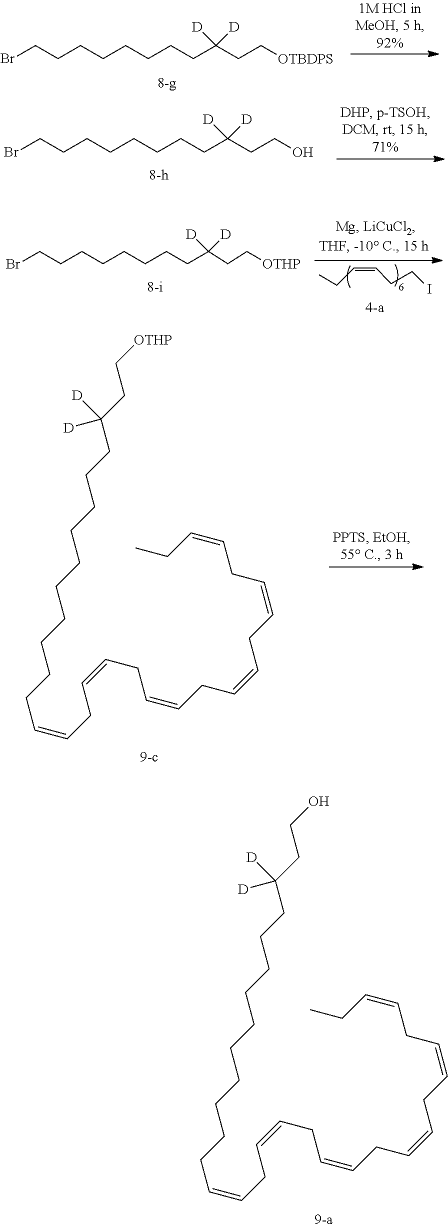

### Step 1: Preparation of 11-bromoundecan-3,3-d2-1-ol (Compound 8-h)

Compound 8-g (3.10 g, 6.31 mmol) was added to a solution of HCl in MeOH (80 mL, 1M). The mixture was stirred at room temperature for 5 h. The resulting reaction was quenched with saturated NaHCO₃ solution (100 mL) and extracted with ether (3×100 mL). The combined ether extracts were washed with H2O, brine and dried over anhydrous MgSO₄. After evaporation of solvents, the residue was purified by silica gel flash chromatography to afford compound 8-h (1.46 g, 92% yield). ¹H NMR (CDCl₃): δ 3.64 (t, 2H, J=6.7 Hz), 3.41 (t, 2H, J=6.8 Hz), 1.85 (m, 2H), 1.58-1.34 (m, 14H).

### Step 2: Preparation of 2-((11-bromoundecyl-3,3-d2)oxy)tetrahydro-2H-pyran (Compound 8-i)

A stirred solution of compound 8-h (1.44 g, 5.71 mmol) in CH₂C12 (60 mL) was added 3,4-dihydropyran (2 mL, 22.8 mmol) followed by p-toluenesulfonic acid monohydrate (86 mg, 0.50 mmol) at 0° C. After stirring for 15 h, the reaction mixture was diluted with CH₂C12 (100 mL) and washed with saturated NaHCO₃ solution, H2O, brine and dried over anhydrous MgSO₄. After evaporation of solvents, the residue was purified by silica gel flash chromatography to afford compound 8-i (1.39 g, 71%). ¹H NMR (CDCl₃): δ 4.57 (m, 1H), 3.87 (m, 1H), 3.73 (m, 1H), 3.49 (m, 1H), 3.41 (t, 2H, J=7.0 Hz), 3.37 (m, 1H), 1.90-1.80 (m, 3H), 1.59-1.48 (m, 8H), 1.43-1.24 (m, 11H).

### Step 3: Preparation of 2-(((14Z,17Z,20Z,23Z,26Z,29Z)-dotriaconta-14,17,20,23,26,29-hexaen-1-yl-3,3-d2)oxy)tetrahydro-2H-pyran (Compound 9-c)

Compound 9-c was prepared by following the same procedure as in Step 2 of Example 1 to prepare compound 5-a.

### Step 4: Preparation of (14Z,17Z,20Z,23Z,26Z,29Z)-dotriaconta-14,17,20,23,26,29-hexaen-3,3-d2-1-ol (Compound 9-a)

Pyridinium p-toluenesulfonateanol was added to the solution of compound 9-c in EtOH. The reaction mixture was heated at 55° C. for 3 h before it was then concentrated under reduced pressure. CH₂C12 was added to the remaining residue and the organic solvents were washed with saturated NaHCO₃ solution, H2O, brine and dried over anhydrous MgSO₄. After evaporation of solvents, the residue was purified by silica gel flash chromatography to afford compound 9-a.

1. A method for synthesizing a very long chain fatty acid comprising
(a) Reacting a protected leaving group-substituted saturated aliphatic group (2) with an halogen-substituted unsaturated aliphatic group (4) to form a protected aliphatic group (5) 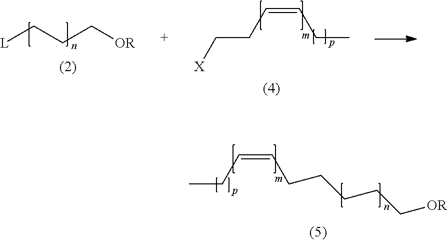
(b) deprotecting the protected aliphatic group (5) to form an alcohol (6) 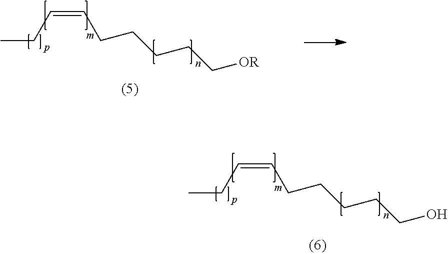
(c) oxidizing the alcohol (6) to form a very long chain fatty acid (7); 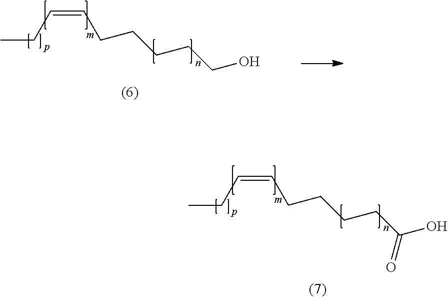
where:
n is an integer from 3 to 14;
m is an integer from 3 to 6;
p is an integer from 1 to 4;
L is a leaving group;
X is halogen; and
R is a protecting group.

2. The method of claim 1, where X is Cl, Br, or I; and p is 1 or 4.

3. The method of claim 1, wherein the leaving group (L) is acetoxy (—OC(═O)CH₃), tosyl (—OTs), nosyl (—ONs), Cl, Br, or I, where L and X are not the same.

4. The method of claim 1, wherein X is I and the leaving group (L) is Br.

5. The method of claim 1, wherein the protecting group is an acetyl, benzoyl, benzyl, β-methoxyethoxyether, methoxymethyl ether, dimethoxytrityl, p-methoxybenzyl ether, p-methoxyphenylether, methythiomethylether, pivaloyl, tetrahydropyranyl, tetrahydrofuranyl, trityl, silyl ethyl, methyl ether, or ethoxyethyl ether protecting group.

6. The method of claim 5, wherein the protecting group is a silyl ether protecting group selected from a trimethyl silyl group (TMS), a tert-butyldimethylsilyl group (TBDMS), tri-iso-propylsilyloxymethyl group (TOM), triisopropylsilyl group (TIPS), or tert-butyldiphenylsilyl group (TBDPS).

7. The method of claim 1, wherein the protected leaving group (L)-substituted saturated aliphatic group (2) is di-deuterated at the β-position and the method provides a very long chain fatty acid di-deuterated at the β-position.

8. The method of claim 1 wherein the very long chain fatty acid (7) is an alpha-linolenic fatty acid which is a 22:6n3, 24:6n3, 26:6n3, 28:6n3, 30:6n3, 32:6n3, 34:6n3, 36:6n3, 38:6n3, 18:3n3, 18:4n3, 20:4n3, 22:5n3, 24:5n3, 26:5n3, 28:5n3, 30:5n3, 32:5n3, 34:5n3, 36:5n3, or 38:5n3 fatty acid.

9. The method of claim 8, wherein the very long chain fatty acid (7) is an alpha-linolenic fatty acid which is a 32:6n3, 34:6n3, 32:5n3, or 34:5n3 fatty acid.

10. The method of claim 1, wherein the very long chain fatty acid (7) is an linolenic fatty acid which is a 18:2n6, 18:3n6, 20:4n6, 22:4n6, 24:4n6, 26:4n6, 28:4n6, 30:4n6, 32:4n6, 34:4n6, 36:4n6, 38:4n6, 22:5n6, 24:5n6, 26:5n6, 28:5n6, 30:5n6, 32:5n6, 34:5n6, 36:5n6, or 38:5n6 fatty acid.

11. The method of claim 10, wherein the very long chain fatty acid (7) is an linolenic fatty acid which is a 28:4n6, 34:4n6, 36:4n6, 30:5n6, or 34:5n6 fatty acid.

12. A deuterated very long chain fatty acid of Formula I 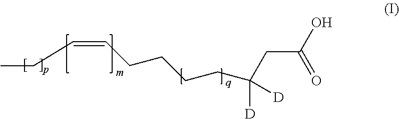
or a pharmaceutically acceptable salt thereof, wherein
m is an integer from 3 to 6;
p is an integer from 1 to 4; and
q is an integer from 2 to 13.

13. The very long chain fatty acid of claim 12, wherein the level of deuteration at each position indicated to be deuterated is greater than 50%.

14. The long chain fatty acid of claim 12, wherein the level of deuteration at each position indicated to be deuterated is greater than 90%.

15. A pharmaceutical composition comprising a deuterated very long chain fatty acid of claim 12 or a salt thereof, together with pharmaceutically acceptable carrier.

16. A method of treating or preventing macular degeneration in a patient comprising administering an effective amount of a compound of claim 12 to the patient.

17. A method of reducing treating or preventing any of the following in a patient: heart failure, type II diabetes, cardiovascular disease, cardiac arrhythmia, cognitive decline, cancer, or hypertension comprising administering an effective amount of a compound of claim 12 to the patient.

18. The method of claim 17, wherein the cardiac arrhythmia is atrial fibrillation or premature ventricular contractions.

19. The method of claim 1, wherein
L, the leaving group, is acetoxy (—OC(═O)CH₃), tosyl (—OTs), nosyl (—ONs), Cl, Br, or I, where L and X are not the same; and
the protecting group is an acetyl, benzoyl, benzyl, β-methoxyethoxyether, methoxymethyl ether, dimethoxytrityl, p-methoxybenzyl ether, p-methoxyphenylether, methythiomethylether, pivaloyl, tetrahydropyranyl, tetrahydrofuranyl, trityl, silyl ethyl, methyl ether, or ethoxyethyl ether protecting group.

20. The method of claim 1, wherein
the very long chain fatty acid (7) is an alpha-linolenic fatty acid chosen from a 22:6n3, 24:6n3, 26:6n3, 28:6n3, 30:6n3, 32:6n3, 34:6n3, 36:6n3, 38:6n3, 18:3n3, 18:4n3, 20:4n3, 22:5n3, 24:5n3, 26:5n3, 28:5n3, 30:5n3, 32:5n3, 34:5n3, 36:5n3, and 38:5n3 fatty acid; or
the very long chain fatty acid (7) is an linolenic fatty acid chosen from a 18:2n6, 18:3n6, 20:4n6, 22:4n6, 24:4n6, 26:4n6, 28:4n6, 30:4n6, 32:4n6, 34:4n6, 36:4n6, 38:4n6, 22:5n6, 24:5n6, 26:5n6, 28:5n6, 30:5n6, 32:5n6, 34:5n6, 36:5n6, and 38:5n6 fatty acid.
